(19) United States
(12) Patent Application Publication
(10) Pub. No.: US 2025/0303140 A1
(43) Pub. Date: Oct. 2, 2025

(54) Apparatus And Method For Array Positioning

(71) Applicant: NOVOCURE GMBH, Baar (CH)

(72) Inventors: Steven Robbins, Wayne, PA (US); SHALOM STRAUSS, Haifa (IL); ELIE YAACOBI, Haifa (IL)

(21) Appl. No.: 19/094,167

(22) Filed: Mar. 28, 2025

Related U.S. Application Data
(60) Provisional application No. 63/571,828, filed on Mar. 29, 2024.

(51) Int. Cl. A61N 1/04

(52) U.S. Cl. CPC A61N 1/0476; A61N 1/0484

(57) ABSTRACT

An apparatus for applying at least one electrode array to a head of an individual is disclosed. The apparatus includes a head receiving structure configured to receive at least a portion of the head of the individual. The head receiving structure defines an interior. The head receiving structure comprises an adhesive or hook or loop material within the interior. The adhesive or hook or loop material is configured to retain the at least one electrode array within the interior of the head receiving structure.

### CROSS-REFERENCE TO RELATED APPLICATIONS

This application claims priority to and the benefit of the filing date of U.S. Provisional Patent Application No. 63/571,828, filed Mar. 29, 2024, the entirety of which is hereby incorporated by reference herein.

### FIELD

This application relates to systems and methods for positioning electrode arrays.

### BACKGROUND

Tumor Treating Fields (TTFields) therapy is a proven approach for treating tumors using alternating electric fields at frequencies between 50 kHz-1 MHz, more commonly, 100-500 kHz. In current commercial systems, the alternating electric fields are induced by electrode assemblies (e.g., arrays of capacitively coupled electrodes, also called transducer arrays or electrode arrays) placed on opposite sides of a target region of the subject's body. When an AC voltage is applied between opposing electrode arrays, an AC current is coupled through the electrode arrays and into the subject's body.

Proper positioning of electrode arrays relative to each other and a target region (e.g., a tumor) can affect performance of treatment. However, proper placement can be difficult, particularly when the subject is placing the electrode arrays on himself/herself. Thus, this difficulty can diminish the independence of the subject, requiring the subject to have another person (helper) position the electrode arrays. Accordingly, a way to assist a subject with properly positioning one or more electrode arrays is desirable.

### SUMMARY

TTFields are approved for the treatment of glioblastoma multiforme (GBM), and may be delivered, for example, via the OPTUNE® system (Novocure GmbH, Baar, Switzerland), which includes transducer arrays placed on the patient's shaved head. More recently, TTFields therapy has been approved as a combination therapy with chemotherapy for malignant pleural mesothelioma (MPM) and non-small cell lung cancer (NSCLC), and may find use in treating tumors in other parts of the body. For example, recent studies have shown that TTFields treatment can reduce progression of metastases to the brain from non-small cell lung cancer (NSCLC).

Disclosed herein, in one aspect, an apparatus for applying at least one electrode array to a head of an individual includes a head receiving structure configured to receive at least a portion of the head of the individual. The head receiving structure defines an interior. The head receiving structure comprises an adhesive or hook or loop material within the interior. The adhesive or hook or loop material is configured to retain the at least one electrode array within the interior of the head receiving structure.

In another aspect, an apparatus includes a head receiving structure configured to receive at least a portion of the head of the individual. The head receiving structure defines an interior. The head receiving structure comprises first and second portions. The first and second portions are coupled together to permit relative movement between the first portion and the second portion. The head receiving structure comprises an adhesive or hook or loop material within the interior. The adhesive or hook or loop material is configured to retain the at least one electrode array within the interior of the head receiving structure.

A system includes an apparatus for applying at least one electrode array to a head of an individual and an electrode placement map receivable within the interior of the head receiving structure of the apparatus. The electrode placement map includes a 2-dimensional substrate. The electrode placement map further includes visible markings associated with the 2-dimensional substrate, the visible markings being indicative of placement locations of a plurality of electrode arrays relative to each other in 2-dimensional space. The placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space.

A method of using an apparatus for applying at least one electrode array to a head of an individual includes positioning within the interior of the head receiving structure at least one electrode array in a position so that when the head receiving structure is positioned on a head of an individual, the at least one electrode array is in a predetermined position relative to the head of the individual.

### BRIEF DESCRIPTION OF THE DRAWINGS

FIG. 1 is a schematic view of a patient with electrode arrays positioned on the patient in an optimal position for treating a target region.

FIG. 2 is a front view of an exemplary apparatus for applying at least one electrode array to a head of an individual as disclosed herein.

FIG. 3 is a side view of the apparatus of FIG. 2.

FIG. 4 is an underside view of the apparatus of FIG. 2.

FIG. 5 is a view of the apparatus of FIG. 2 in a configuration in which opposed portions of the head receiving structure of the apparatus are pivoted away from each other.

FIG. 6 is a schematic view of the apparatus placed on the head of the patient.

FIG. 7 is a schematic view of an exemplary electronic 2-dimensional map based on an electronic 3-dimensional map of the arrangement of electrode arrays shown in FIG. 1.

FIG. 8 is a top plan view of an exemplary electrode placement map based on the electronic 2-dimensional map of FIG. 7.

FIG. 9 is a top plan view of an exemplary assembly of coupled electrode arrays arranged based on the exemplary electrode placement map of FIG. 8.

FIG. 10 is a cross-sectional schematic side view of an exemplary treatment assembly comprising an electrode array.

Various embodiments are described in detail below with reference to the accompanying drawings, wherein like reference numerals represent like elements, and wherein descriptions of like elements may not be repeated for every embodiment, but may be considered to be the same if previously described herein.

### DESCRIPTION OF THE PREFERRED EMBODIMENTS

As discussed above, proper placement of electrode arrays can be difficult, particularly when the subject is placing the electrode arrays on himself/herself. This application relates to the accurate positioning of electrode arrays that may be used, e.g., for delivering TTFields to a subject's body (for example, the head) and treating one or more cancers or tumors located in the subject's body (e.g., the head). The subject may then reproduce the accurate positioning of the electrode arrays for future application of the arrays on the head.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Any combination of the elements described herein in all possible variations thereof is encompassed by the invention unless otherwise indicated herein or otherwise clearly contradicted by context.

As used in the specification and the appended claims, the singular forms “a,” “an,” and “the” include plural referents unless the context clearly dictates otherwise. Thus, it is contemplated that disclosure of a singular form of an element can provide support for embodiments in which only a single such element is provided, as well as support for embodiments in which a plurality of such elements are provided.

As used herein, a “fiducial” can be understood as referring to an object in the field of view of an imaging system that appears in the image produced and that can be used as a point of reference or a measure. Optionally, a “fiducial” can be an anatomical feature of a body of a patient, such as a facial feature (e.g., a nose, an ear, an eye, or an eyebrow, etc.).

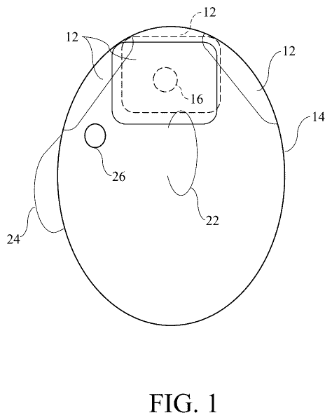

FIG. 1 illustrates a plurality of electrode arrays 12 on a head 14 of an individual, illustrating an exemplary set of ideal placement locations of the plurality of electrode arrays optimized for positioning relative to a target region 16 for delivering tumor-treating fields.

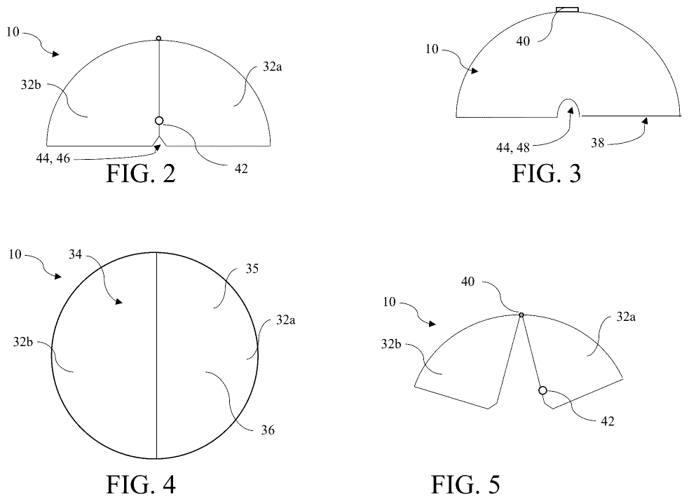

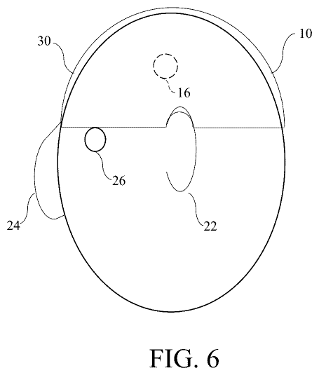

Disclosed herein, with reference to FIGS. 1-6, is an apparatus 10 for applying at least one electrode array 12 to a head 14 of an individual. The apparatus 10 comprises a head receiving structure 30 configured to receive at least a portion of the head 14 of the individual. The head receiving structure 30 can define an interior 34. The head receiving structure 30 can comprise an adhesive or hook or loop material 36 within the interior 34. The adhesive or hook or loop material 36 can be configured to retain the at least one electrode array 12 within the interior 34 of the head receiving structure 30.

In exemplary aspects, the adhesive or hook or loop material 36 can cover at least 10%, at least 20%, at least 40%, or at least 50%, or at least 60%, or at least 70%, or at least 80%, or at least 90% or substantially all or all of an area defined by an interior surface 35 of the head receiving structure 30.

In some optional aspects, the head receiving structure 30 can have first and second portions 32a,b. The first and second portions 32a,b can be coupled together to permit relative movement between the first portion and the second portion.

In exemplary aspects, the head receiving structure 30 can comprise a hinge or linkage 40 that couples the first and second portions 32a,b to permit pivotal movement therebetween. For example, the hinge or linkage 40 can be a hinge that comprises a leaf, a knuckle, and a pin that couples the leaf to the knuckle. In other aspects, the hinge or linkage 40 can comprise a flexible material that extends between and couples to each of the first and second portions 32a,b of the head receiving structure. For example, the hinge or linkage 40 can be a hinge. In further optional aspects, the hinge can be a living hinge. In additional optional aspects, the hinge or linkage can be a linkage such as, for example, a flexible material such as a flexible polymer, an elastic material, or a textile. In additional aspects, the linkage can comprise a plurality of linked elements, wherein coupled linked elements of the plurality of linked elements are linked about respective pivotal axes. More generally, the hinge or linkage 40 can be any suitable connection that permits pivotal movement between the first and second portions 32a,b.

In some aspects, the head receiving structure 30 can comprise a fastener 42 that couples the first and second portions 32a,b together. The fastener 42 can be configured to permit selective decoupling between the first and second portions 32a,b to thereby permit relative movement between the first portion and the second portion. In various aspects, the fastener 42 can comprise, for example, hook and loop material, a zipper, a button, or a snap, or the like, or combinations thereof.

In various aspects, the first and second portions 32a,b of the head receiving structure 30 can be substantially rigid. In other aspects, one or both of the first portion 32a and the second portion 32b of the head receiving structure 30 can be flexible. For example, the first portion 32a and/or the second portion 32b of the head receiving structure 30 can be resiliently elastic. In other aspects, one or both of the first portion 32a and the second portion 32b of the head receiving structure 30 can be flexible but not elastic. For example, one or both of the first portion 32a and the second portion 32b of the head receiving structure 30 can comprise cloth or fabric.

In some aspects, each of the first and second portions 32a,b of the head receiving structure 30 form approximately half (e.g., 40% to 60% of the surface area and/or volume) of the head receiving structure 30.

In some aspects, optionally, the head receiving structure 30 need not comprise first and second portions that are coupled together to permit relative movement between the first portion and the second portion. For example, in some optional aspects, the head receiving component can be a unitary structure. In some aspects, the head receiving structure 30 can be flexible. For example, the head receiving structure 30 can be flexible and resiliently elastic. The head receiving structure 30 can be a beanie or skull cap. Optionally, in these aspects, the head receiving structure can be removed from the head by rolling and/or folding the head receiving structure upwardly and away from the head. In other aspects, the head receiving structure 30 can be flexible but not elastic. For example, the head receiving structure 30 can comprise cloth or fabric. The head receiving structure 30 can be a cap (similar to a baseball cap or cricket cap). For example, the head receiving structure 30 can have the shape of a cap and materials that are the same as, or perform in a similar manner to, a conventional cap.

The apparatus 10 can further comprise at least one orientation element 44 that is configured to cooperate with a respective fiducial to orient the apparatus relative to the head of the individual. In some aspects, the respective fiducial can be a facial feature. For example, the respective facial feature can be a nose 24, an ear 22, an eye 26, or an eyebrow. In some aspects, the head receiving structure 30 can have a lower perimeter 38 having a shape. The at least one orientation element 44 can comprise at least one of: a protrusion extending into the interior, a marking, or a feature that alters the shape of the lower perimeter. For example, the at least one orientation element 44 can incorporate or otherwise account for the shape of the feature in the shape of the lower perimeter. Said feature can comprise, for example, a recess or a downwardly extending projection. As illustrated in FIG. 2, in some aspects, the head receiving structure 30 can comprise a first orientation element 44 defined by a recess 46 in the lower perimeter 38 of the head receiving structure 30 for receiving a portion of the nose 24. A second orientation element 44 can include a recess 46 in the lower perimeter 38 of the head receiving structure 30 for receiving a portion of an ear 22. In some aspects, the head receiving structure 30 can define a pair of cutouts 48 configured to receive respective ears 22 of the individual. Each of these orientation elements 44 can help the subject re-position the head receiving structure 30 onto the head in a repeatable manner so that, once an optimized set of locations for the arrays has been established, the same relative positions for the arrays can be repeated for future applications of the arrays in a relatively easy manner without the aid of a helper. And two or more orientation elements 44 can ensure greater precision in positioning the head receiving structure 30.

In some aspects, the adhesive or hook or loop material 36 can be selected to have a first adhesion between the head receiving structure and the at least one electrode array that is less than a second adhesion between an adhesive of the at least one electrode array 12 and the head 14 of the individual.

In exemplary aspects, the head receiving structure 30 can be embodied as a helmet. In other aspects, the head receiving structure 30 can be embodied as a hat or cap or beanie.

A method of using the apparatus 10 can include positioning at least one electrode array 12 within, and coupled to, the head receiving structure 30 so that when the head receiving structure is positioned on the head 14 of an individual, the at least one electrode array is in a predetermined position (a preferred position) relative to the head of the individual. The preferred position, including both the location on the head and the orientation (relative to the head and relative to other arrays), may be determined from an MRI or CT scan image, with the help of specialty software (such as MAXPOINT™ treatment planning software from Novocure). The MAXPOINT™ treatment planning software includes software to convert a 3D image obtained from an MRI or CT scan into a 2D image. The head receiving structure 30 can be placed over the head 14 of the individual to position the electrode array(s) 12 thereon.

The head receiving structure 30 can be removed from the head 14 of the individual, leaving the at least one electrode array 12 attached to the head of the individual in the preferred position (e.g., via the adhesive of the at least one electrode array 12). In aspects in which the head receiving structure 30 comprises first and second portions 32a,b and a hinge or linkage 40 that couples the first and second portions to permit pivotal movement therebetween, removing the head receiving structure from the head of the individual can comprise pivoting the first portion 32a relative to the second portion 32b about the hinge or linkage 40. In aspects in which the head receiving structure is flexible, removing the head receiving structure from the head of the individual can comprise rolling the head receiving structure from the head of the individual. In this manner, the head receiving structure 30 can function as an array applicator.

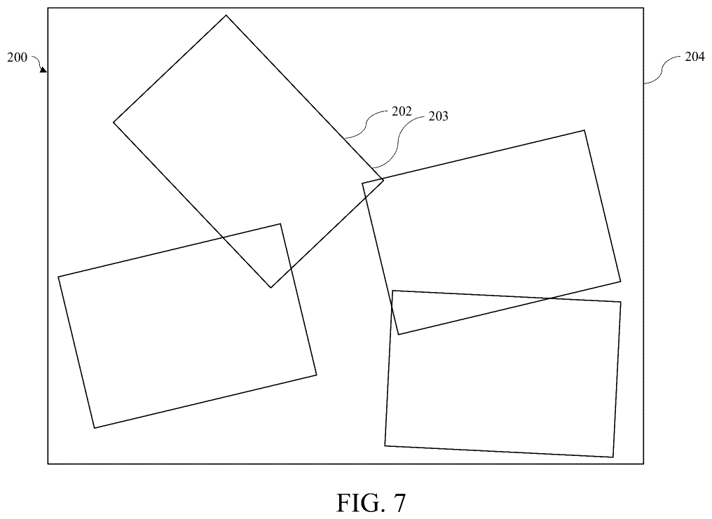

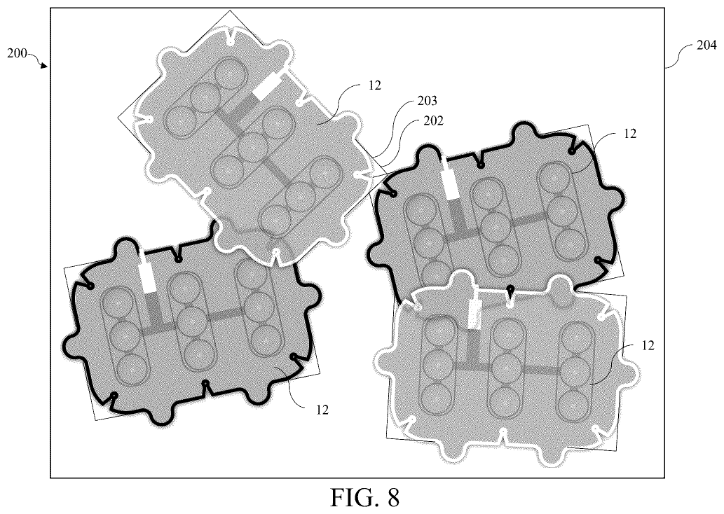

In some aspects, and with reference to FIGS. 7-8, an electrode placement map 200 can be positioned within the interior 34 of the head receiving structure 30 of the apparatus 10 for guiding placement of the electrode arrays within the apparatus. The electrode placement map 200 can comprise a 2-dimensional substrate 204 and visible markings 203 of placement locations 202 associated with the 2-dimensional substrate. The visible markings 203 can be indicative of placement locations of a plurality of electrode arrays 12 relative to each other in 2-dimensional space. The placement locations of the plurality of electrode arrays 12 relative to each other in 2-dimensional space can translate to optimized positions of the plurality of electrode arrays for delivering tumor-treating fields when the plurality of electrode arrays are placed on a patient. The plurality of electrode arrays 12 can be positioned within the interior 34 of the head receiving structure 30 based on the visible markings of the electrode placement map. The same types of orientation elements 44 discussed above can be included on the electrode placement map 200 with respect to relative locations of fiducials in order to ascertain the preferred locations of the arrays 12 within the head receiving structure 30. In some aspects, the electrode arrays 12 can attach to the electrode placement map 200 (e.g., via adhesive or hook and loop material). For example, in these aspects, the electrode arrays can comprise adhesive on an outer surface of the electrode arrays 12 opposite the surface configured to face the patient. Optionally, said adhesive can cover only a portion of the outer surface (e.g., in one small region or a plurality of spaced regions). In other aspects, the map can merely serve as an indicator for electrode placement, and the electrode arrays need not couple to the electrode placement map 200.

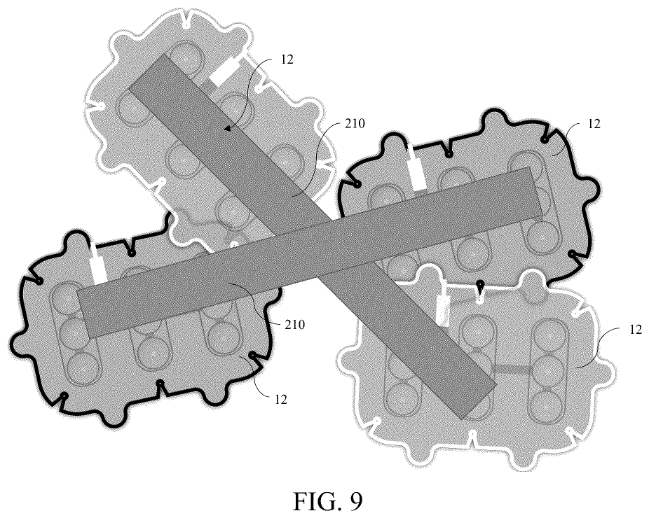

Referring to FIGS. 8-9, in some aspects, the electrode placement map 200 can be used to position the electrode arrays 12. The electrode arrays 12 can be placed on the electrode placement map 200 according to the markings 203. Connecting strips 210 (e.g., comprising adhesive or hook or loop material) can be used to secure the electrode arrays 12 together. For example, a first strip of the connecting strips 210 can couple a first pair of electrode arrays 12, and a second strip of the connecting strips 210 can couple a second pair of electrode arrays so that the first and second strips cross and couple together. In this way, the electrode arrays 12 can be coupled together as a unit. Optionally, hook and loop material can be used both on the surface of the electrode placement map 200 and on the outward-facing side of the electrode arrays 12 (facing away from the skin) to temporarily hold them together. The skin-facing side of the arrays faces away from the electrode placement map 200, and areas of exposed adhesive (that will be used to adhere to the skin) can be protected by a release liner (or release liners). Once properly positioned (according to the electrode placement map 200), the release liner(s), if present, may then be removed. The electrode arrays (e.g., two pairs coupled together) and the electrode placement map 200 can then be positioned on the head of the user, with the electrode arrays 12 between the electrode placement map and the head. The electrode placement map 200 can be peeled away to leave the electrode arrays 12 adhered on the head. This can provide an initial array location for the patient. In some aspects, the outward-facing side of the electrode placement map 200 may have an adhesive or hook and loop material to enable attachment to the interior of the head receiving structure 30 (which may have a complementary hook and loop material). In this scenario, the electrode placement map 200 can be peeled away to leave the electrode arrays 12 adhered on the head and the electrode placement map 200 within the head receiving structure 30. The apparatus 10 can then be used to repeat the placement for subsequent applications of the electrode arrays 12.

In some aspects, the apparatus 10 can be used in a system further comprising an electrode placement map 200 receivable within the interior 34 of the head receiving structure 30 of the apparatus 10. The electrode placement map 200 can comprise a 2-dimensional substrate 204 and visible markings 203 of placement locations 202 associated with the 2-dimensional substrate. The visible markings 203 can be indicative of placement locations of a plurality of electrode arrays 12 relative to each other in 2-dimensional space. The placement locations of the plurality of electrode arrays 12 relative to each other in 2-dimensional space can translate to optimized positions of the plurality of electrode arrays for delivering tumor-treating fields when the plurality of electrode arrays are placed on a patient.

For example, the 2-dimensional substrate 204 can comprise paper, cloth or fabric. In some optional aspects, the visible markings 203 can comprise ink or toner printed on the 2-dimensional substrate 204.

In some aspects, the visible markings 203 can comprise indications of at least a portion of a perimeter of each electrode array of the plurality of electrode arrays. For example, as illustrated, the visible markings 203 can comprise rectangles that generally correspond to portions of the outer perimeter of the electrode arrays. Optionally, as shown in FIGS. 7-8, the visible markings 203 and the electrode arrays 12 can have corresponding edges that can be at least partially aligned with each other. For example, the visible markings 203 can define rectangles having straight edges, and the outer perimeter of a corresponding electrode array 12 can have at least some edge portions that can be aligned or substantially aligned with a straight edge of a corresponding rectangle of the electrode placement map. Optionally, it is contemplated that the electrode array 12 can also include edge portions that are positioned inwardly or outwardly of a corresponding rectangle of the electrode placement map. In other aspects, the visible markings 203 can comprise geometry that traces the exact outer perimeter (or a portion thereof) of a particular array. For example, in some optional aspects, the indications of at least a portion of a perimeter of each electrode array of the plurality of electrode arrays comprises an outline of at least a portion of the perimeter of each electrode array of the plurality of electrode arrays. In still other aspects, the visible markings 203 can comprise reference points that can correspond to (and can be aligned with) reference points on the outer perimeter of the electrode arrays. For example, reference points corresponding to reference points on the outer perimeter of the electrode arrays can include one or more identified fiducials in such instances that the fiducial(s) can help with relative spacing and orientation of the electrode arrays. Optionally, these reference points can be included in the 2-dimensional electrode placement map 200. In other aspects, the visible markings 203 can comprise crosshairs (or some similar indication) that can indicate a centroid of an electrode array. More generally, the visible markings 203 can provide indications that a user can reference to provide an indication of a placement location and, optionally, an orientation of an electrode array.

Additional details of exemplary electrode placement maps and systems and methods for providing and using electrode placement maps are disclosed in U.S. patent application Ser. No. 18/490,315, filed Oct. 19, 2023, the entirety of which is hereby incorporated by reference herein for all purposes.

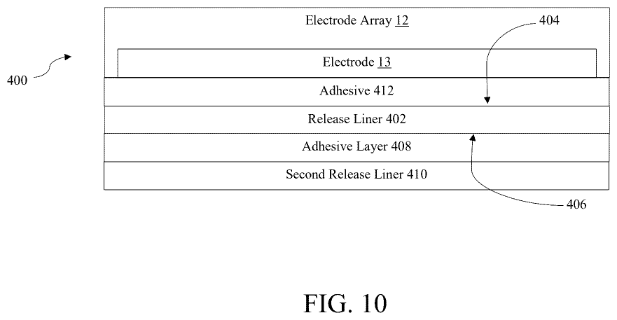

Referring to FIG. 10, a treatment assembly 400 can be used for initially positioning one or more electrode arrays 12, wherein the treatment assembly 400 permits detachment from the patient to confirm the proper positioning of the electrode array(s) 12. The treatment assembly 400 can comprise an electrode array 12 comprising at least one electrode 13 which may be accompanied by a layer of adhesive 412 on the skin-facing surface of the electrode. A first release liner 402 can be used to temporarily cover this layer of adhesive 412. The release liner 402 can comprise a first surface 404 facing the electrode array 12 and a second surface 406 opposite the first surface. A second adhesive layer 408 can be aligned on the second surface 406 of the first release liner. In some aspects, a second release liner 410 can be positioned over the adhesive layer 408.

After removal of second release liners 410 (if present), a plurality of the treatment assemblies 400 can be positioned on the head of the patient so that the adhesive layer 408 adheres the plurality of the treatment assemblies to the head of the patient. An electrode placement map 200 can be applied over the plurality of the treatment assemblies 400. The electrode placement map 200 can comprise an adhesive or hook or loop material that adheres to the plurality of the treatment assemblies so that the treatment assemblies attach thereto. The electrode placement map with the treatment assembly coupled thereto can be removed from the head of the patient. The positions of the plurality of treatment assemblies 400 can be confirmed using the electrode placement map. Once the positions of the plurality of treatment assemblies are confirmed, the release liners 402 can be removed from the treatment assemblies 400 to expose the layer of adhesive 412 of the electrode arrays 12. The electrode arrays 12 can be applied to the patient while the electrode arrays are adhered to the electrode placement map so that the adhesive of the electrode array adheres to the patient.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus for applying at least one electrode array to a head of an individual, the apparatus comprising:
a head receiving structure configured to receive at least a portion of the head of the individual, the head receiving structure comprising first and second portions, wherein the first and second portions are coupled together to permit relative movement between the first portion and the second portion,
wherein the head receiving structure defines an interior, wherein the head receiving structure comprises an adhesive or hook or loop material within the interior, and wherein the adhesive or hook or loop material is configured to retain the at least one electrode array within the interior of the head receiving structure.

Aspect 2: The apparatus of aspect 1, wherein the head receiving structure comprises a hinge or linkage that couples the first and second portions to permit pivotal movement therebetween.

Aspect 3: The apparatus of aspect 2, wherein the hinge or linkage is a hinge that comprises a leaf, a knuckle, and a pin that couples the leaf to the knuckle.

Aspect 4: The apparatus of aspect 2, wherein the hinge or linkage is a hinge that comprises a flexible material that extends between and couples to each of the first and second portions of the head receiving structure.

Aspect 5: The apparatus of aspect 4, wherein the hinge is a living hinge.

Aspect 6: The apparatus of aspect 1, wherein the head receiving structure comprises a fastener that couples the first and second portions together, wherein the fastener is configured to permit selective decoupling between the first and second portions to thereby permit relative movement between the first portion and the second portion.

Aspect 7: The apparatus of aspect 6, wherein the fastener comprises hook and loop material, a zipper, a button, or a snap.

Aspect 8: The apparatus of any one of the preceding aspects, wherein the first and second portions of the head receiving structure are substantially rigid.

Aspect 9: The apparatus of any one of aspects 1-7, wherein at least one of the first portion or the second portion of the head receiving structure is flexible.

Aspect 10: The apparatus of aspect 9, wherein the at least one of the first portion or the second portion of the head receiving structure that is flexible is resiliently elastic.

Aspect 11: The apparatus of any one of the preceding aspects, wherein each of the first and second portions of the head receiving structure form approximately half of the head receiving structure.

Aspect 12: The apparatus of any one of the preceding aspects, wherein the adhesive or hook or loop material is selected to have a first adhesion between the head receiving structure and the at least one electrode array that is less than a second adhesion between an adhesive of the at least one electrode array and the head of the individual.

Aspect 13: The apparatus of any one of the preceding aspects, further comprising at least one orientation element that is configured to cooperate with a respective fiducial to orient the apparatus relative to the head of the individual.

Aspect 14: The apparatus of aspect 13, wherein the respective fiducial is a facial feature.

Aspect 15: The apparatus of aspect 14, wherein the respective facial feature is a nose, an ear, an eye, or an eyebrow.

Aspect 16: The apparatus of any one of aspects 13-15, wherein the head receiving structure comprises a lower perimeter having a shape, wherein the at least one orientation element comprises at least one of: a protrusion extending into the interior, a marking, or a feature in the shape of the lower perimeter.

Aspect 17: The apparatus of aspect 16, wherein the at least one orientation element comprises the feature in the shape of the lower perimeter, and wherein the feature comprises a recess or a downwardly extending projection.

Aspect 18: The apparatus of any one of the preceding aspects, wherein the head receiving structure comprises a pair of cutouts configured to receive respective ears of the individual.

Aspect 19: An apparatus for applying at least one electrode array to a head of an individual, the apparatus comprising:
a head receiving structure configured to receive at least a portion of the head of the individual, wherein the head receiving structure defines an interior, wherein the head receiving structure comprises an adhesive or hook or loop material within the interior of the head receiving structure, and wherein the adhesive or hook or loop material is configured to retain the at least one electrode array within the interior of the head receiving structure.

Aspect 20: The apparatus of aspect 19, wherein the head receiving structure is flexible.

Aspect 21: The apparatus of aspect 20, wherein the head receiving structure that is flexible is resiliently elastic.

Aspect 22: The apparatus of any one of aspects 19-21, wherein the adhesive or hook or loop material is selected to have a first adhesion between the head receiving structure and the at least one electrode array that is less than a second adhesion between an adhesive of the at least one electrode array and the head of the individual.

Aspect 23: The apparatus of any one of aspects 19-22, further comprising at least one orientation element that is configured to cooperate with a respective fiducial to orient the apparatus relative to the head of the individual.

Aspect 24: The apparatus of aspect 23, wherein the respective fiducial is a facial feature.

Aspect 25: The apparatus of aspect 24, wherein the respective facial feature is a nose, an ear, an eye, or an eyebrow.

Aspect 26: The apparatus of any one of aspects 23-25, wherein the head receiving structure comprises a lower perimeter having a shape, wherein the at least one orientation element comprises at least one of: a protrusion extending into the interior, a marking, or a feature in the shape of the lower perimeter.

Aspect 27: The apparatus of aspect 26, wherein the at least one orientation element comprises the feature in the shape of the lower perimeter, and wherein the feature comprises a recess or a downwardly extending projection.

Aspect 28: The apparatus of any one of aspects 19-27, wherein the head receiving structure comprises a pair of cutouts configured to receive respective ears of the individual.

Aspect 29: A system comprising:
an apparatus as in any one of the preceding aspects; and
an electrode placement map receivable within the interior of the head receiving structure of the apparatus, the electrode placement map comprising:
   a 2-dimensional substrate;
   visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space.

Aspect 30: The system of aspect 29, wherein the visible markings of the electrode placement map comprise ink or toner printed on the 2-dimensional substrate.

Aspect 31: The system of aspect 30, wherein the 2-dimensional substrate comprises paper, cloth, or fabric.

Aspect 32: The system of any one of aspects 29-31, wherein the visible markings of the electrode placement map comprise indications of at least a portion of a perimeter of each electrode array of the plurality of electrode arrays.

Aspect 33: The system of any one of aspects 29-32, wherein the visible markings of the electrode placement map comprise an outline of the perimeter of each electrode array of the plurality of electrode arrays.

Aspect 34: The system of any one of aspects 29-33, wherein the visible markings comprise indications of at least one fiducial that provides a reference location from which the plurality of electrode arrays can be positioned.

Aspect 35: A method of using the apparatus as in any one of aspects 1-28, the method comprising:
positioning within the interior of the head receiving structure at least one electrode array in a position so that when the head receiving structure is positioned on a head of an individual, the at least one electrode array is in a predetermined position relative to the head of the individual.

Aspect 36: The method of aspect 35, further comprising placing the head receiving structure over the head of the individual.

Aspect 37: The method of aspect 35, further comprising removing the head receiving structure from the head of the individual, leaving the at least one electrode array attached to the head of the individual.

Aspect 38: The method of aspect 37, wherein the head receiving structure comprises a first and second portion and a hinge or linkage that couples the first and second portions to permit pivotal movement therebetween, wherein removing the head receiving structure from the head of the individual comprises pivoting the first portion relative to the second portion about the hinge or linkage.

Aspect 39: The method of aspect 37, wherein the head receiving structure is flexible, and wherein removing the head receiving structure from the head of the individual comprises rolling the head receiving structure from the head of the individual.

Aspect 40: The method of aspect 35, further comprising, positioning within the interior of the head receiving structure an electrode placement map receivable therein, the electrode placement map comprising:
a 2-dimensional substrate;
visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space,

wherein positioning within the interior of the head receiving structure at least one electrode array comprises positioning a plurality of electrode arrays based on the visible markings of the electrode placement map.

Aspect 41: A treatment assembly comprising:
an electrode array comprising at least one electrode; and
a release liner having:
   a first surface facing the electrode array; and
   a second surface opposite the first surface, wherein the second surface comprises an adhesive.

Aspect 42: A method of using a plurality of treatment assemblies of aspect 41, the method comprising:
positioning the plurality of the treatment assemblies on the head of the patient so that the adhesive of the release liner adheres the plurality of the treatment assemblies to the head of the patient;
placing, on the treatment assemblies so that the treatment assemblies attach thereto, an electrode placement map comprising:
   a 2-dimensional substrate;
   visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space; and
   an adhesive or hook or loop material that adheres to the plurality of the treatment assemblies;
removing the electrode placement map with the treatment assembly attached thereto; and
confirming the position of the plurality of treatment assemblies using the electrode placement map;
removing the release liners from each of the plurality of the treatment assemblies;
applying the electrode arrays to the patient while the electrode arrays are adhered to the electrode placement map.

Aspect 43: An apparatus for applying at least one electrode array to a head of an individual, the apparatus comprising:
a head receiving structure configured to receive at least a portion of the head of the individual,
wherein the head receiving structure defines an interior, wherein the head receiving structure comprises an adhesive or hook or loop material within the interior, and wherein the adhesive or hook or loop material is configured to retain the at least one electrode array within the interior of the head receiving structure.

Aspect 44: The apparatus of aspect 43, wherein the head receiving structure comprises first and second portions, wherein the first and second portions are coupled together to permit relative movement between the first portion and the second portion.

Aspect 45: The apparatus of aspect 44, wherein the head receiving structure comprises a hinge or linkage that couples the first and second portions to permit pivotal movement therebetween.

Aspect 46: The apparatus of aspect 45, wherein the hinge or linkage is a linkage that comprises a fastener that couples the first and second portions together, wherein the fastener is configured to permit selective decoupling between the first and second portions to thereby permit relative movement between the first portion and the second portion.

Aspect 47: The apparatus of aspect 46, wherein the fastener comprises hook and loop material, a zipper, a button, or a snap.

Aspect 48: The apparatus of aspect 44, wherein the first and second portions of the head receiving structure are substantially rigid.

Aspect 49: The apparatus of aspect 44, wherein at least one of the first portion or the second portion of the head receiving structure is flexible.

Aspect 50: The apparatus of aspect 44, wherein each of the first and second portions of the head receiving structure forms approximately half of the head receiving structure.

Aspect 51: The apparatus of aspect 42, wherein the adhesive or hook or loop material is selected to have a first adhesion between the head receiving structure and the at least one electrode array that is less than a second adhesion between an adhesive of the at least one electrode array and the head of the individual.

Aspect 52: The apparatus of aspect 42, further comprising at least one orientation element that is configured to cooperate with a respective fiducial to orient the apparatus relative to the head of the individual.

Aspect 53: The apparatus of aspect 52, wherein the respective fiducial is a facial feature.

Aspect 54: The apparatus of aspect 42, wherein the head receiving structure is flexible.

Aspect 55: The apparatus of aspect 54, wherein the head receiving structure that is flexible is resiliently elastic.

Aspect 56: A system comprising:
an apparatus comprising:
   a head receiving structure configured to receive at least a portion of the head of the individual,
   wherein the head receiving structure defines an interior, wherein the head receiving structure comprises an adhesive or hook or loop material within the interior, and wherein the adhesive or hook or loop material is configured to retain the at least one electrode array within the interior of the head receiving structure; and an electrode placement map receivable within the interior of the head receiving structure of the apparatus, the electrode placement map comprising:
   a 2-dimensional substrate;
   visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space.

Aspect 57: The system of aspect 56, wherein the visible markings of the electrode placement map comprise ink or toner printed on the 2-dimensional substrate.

Aspect 58: The system of aspect 56, wherein the visible markings of the electrode placement map comprise indications of at least a portion of a perimeter of each electrode array of the plurality of electrode arrays.

Aspect 59: The system of aspect 56, wherein the visible markings comprise indications of at least one fiducial that provides a reference location from which the plurality of electrode arrays can be positioned.

Aspect 60: A method comprising:
positioning within an interior of a head receiving structure at least one electrode array in a position so that when the head receiving structure is positioned on a head of an individual, the at least one electrode array is in a predetermined position relative to the head of the individual, wherein the head receiving structure is configured to receive at least a portion of the head of the individual, wherein the head receiving structure defines an interior, wherein the head receiving structure comprises an adhesive or hook or loop material within the interior, and wherein the adhesive or hook or loop material is configured to retain the at least one electrode array within the interior of the head receiving structure.

Aspect 61: The method of aspect 60, wherein the head receiving structure comprises first and second portions and a hinge or linkage that couples the first and second portions to permit pivotal movement therebetween, the method further comprising removing the head receiving structure from the head of the individual by pivoting the first portion relative to the second portion about the hinge or linkage.

Aspect 62: The method of aspect 60, further comprising, positioning within the interior of the head receiving structure an electrode placement map receivable therein, the electrode placement map comprising:
a 2-dimensional substrate;
visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space,

wherein positioning within the interior of the head receiving structure at least one electrode array comprises positioning a plurality of electrode arrays based on the visible markings of the electrode placement map.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying at least one electrode array to a head of an individual, the apparatus comprising:
a head receiving structure configured to receive at least a portion of the head of the individual,
wherein the head receiving structure defines an interior, wherein the head receiving structure comprises an adhesive or hook or loop material within the interior, and wherein the adhesive or hook or loop material is configured to retain the at least one electrode array within the interior of the head receiving structure.

2. The apparatus of claim 1, wherein the head receiving structure comprises first and second portions, wherein the first and second portions are coupled together to permit relative movement between the first portion and the second portion.

3. The apparatus of claim 2, wherein the head receiving structure comprises a hinge or linkage that couples the first and second portions to permit pivotal movement therebetween.

4. The apparatus of claim 3, wherein the hinge or linkage is a linkage that comprises a fastener that couples the first and second portions together, wherein the fastener is configured to permit selective decoupling between the first and second portions to thereby permit relative movement between the first portion and the second portion.

5. The apparatus of claim 4, wherein the fastener comprises hook and loop material, a zipper, a button, or a snap.

6. The apparatus of claim 2, wherein the first and second portions of the head receiving structure are substantially rigid.

7. The apparatus of claim 2, wherein at least one of the first portion or the second portion of the head receiving structure is flexible.

8. The apparatus of claim 2, wherein each of the first and second portions of the head receiving structure forms approximately half of the head receiving structure.

9. The apparatus of claim 1, wherein the adhesive or hook or loop material is selected to have a first adhesion between the head receiving structure and the at least one electrode array that is less than a second adhesion between an adhesive of the at least one electrode array and the head of the individual.

10. The apparatus of claim 1, further comprising at least one orientation element that is configured to cooperate with a respective fiducial to orient the apparatus relative to the head of the individual.

11. The apparatus of claim 10, wherein the respective fiducial is a facial feature.

12. The apparatus of claim 1, wherein the head receiving structure is flexible.

13. The apparatus of claim 12, wherein the head receiving structure that is flexible is resiliently elastic.

14. A system comprising:
an apparatus comprising:
a head receiving structure configured to receive at least a portion of the head of the individual,
wherein the head receiving structure defines an interior, wherein the head receiving structure comprises an adhesive or hook or loop material within the interior, and wherein the adhesive or hook or loop material is configured to retain the at least one electrode array within the interior of the head receiving structure; and an electrode placement map receivable within the interior of the head receiving structure of the apparatus, the electrode placement map comprising:
a 2-dimensional substrate; and
visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space.

15. The system of claim 14, wherein the visible markings of the electrode placement map comprise ink or toner printed on the 2-dimensional substrate.

16. The system of claim 14, wherein the visible markings of the electrode placement map comprise indications of at least a portion of a perimeter of each electrode array of the plurality of electrode arrays.

17. The system of claim 14, wherein the visible markings comprise indications of at least one fiducial that provides a reference location from which the plurality of electrode arrays can be positioned.

18. A method comprising:
positioning within an interior of a head receiving structure at least one electrode array in a position so that when the head receiving structure is positioned on a head of an individual, the at least one electrode array is in a predetermined position relative to the head of the individual, wherein the head receiving structure is configured to receive at least a portion of the head of the individual, wherein the head receiving structure defines an interior, wherein the head receiving structure comprises an adhesive or hook or loop material within the interior, and wherein the adhesive or hook or loop material is configured to retain the at least one electrode array within the interior of the head receiving structure.

19. The method of claim 18, wherein the head receiving structure comprises first and second portions and a hinge or linkage that couples the first and second portions to permit pivotal movement therebetween, the method further comprising removing the head receiving structure from the head of the individual by pivoting the first portion relative to the second portion about the hinge or linkage.

20. The method of claim 18, further comprising, positioning within the interior of the head receiving structure an electrode placement map receivable therein, the electrode placement map comprising:
a 2-dimensional substrate; and
visible markings associated with the 2-dimensional substrate, the visible markings being indicative of placement locations of a plurality of electrode arrays relative to each other in 2-dimensional space, wherein the placement locations of the plurality of electrode arrays relative to each other in 2-dimensional space translates to optimized positions of the plurality of electrode arrays for delivering tumor-treating fields when the plurality of electrode arrays are placed on the individual in 3-dimensional space,
wherein positioning within the interior of the head receiving structure at least one electrode array comprises positioning a plurality of electrode arrays based on the visible markings of the electrode placement map.
